(19) United States
(12) Patent Application Publication
(10) Pub. No.: US 2004/0247620 A1
(43) Pub. Date: Dec. 9, 2004

(54) Transformation system based on the integrase gene and attachment site for Myxococcus xanthus bacteriophage Mx9

(75) Inventor: Bryan Julien, Oakland, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kosan Biosciences, Inc., Hayward, CA

(21) Appl. No.: 10/645,818

(22) Filed: Aug. 20, 2003

Related U.S. Application Data
(60) Provisional application No. 60/405,196, filed on Aug. 21, 2002.

(51) Int. Cl. A61K 39/02; C12N 15/74; C12N 1/21

(52) U.S. Cl. 424/200.1; 435/471; 435/252.3

(57) ABSTRACT

The invention provides a transformation system based on bacteriophage Mx9, a temperate phage that infects Myxococcus xanthus. Vectors containing an integrase-encoding gene and a phage attachment site (attP) integrate into a chromosomal attB site and can be used to alter or introduce genes into a variety of host cells.

### CROSS-REFERENCE TO RELATED APPLICATIONS

This application claims benefit of U.S. provisional patent application No. 60/405,196, filed Aug. 21, 2002, the entire contents of which are incorporated by reference.

### FIELD OF THE INVENTION

The invention relates to methods and materials for transforming host bacterial cells using a bacteriophage Mx9 system. The invention finds application in the fields of molecular biology and drug development.

### BACKGROUND OF THE INVENTION

Mx9 is a general transducing phage that infects the Gram-negative bacterium Myxococcus xanthus (9). The phage particle has a polyhedral head with a very short tail. Structurally it resembles Mx8, which also infects M. xanthus.

The integrase gene and attachment site for Mx8 have been characterized (7, 8, 11). Integration of Mx8 by site-specific recombination requires a single phage protein, Int, and the phage attachment site, attP. Unlike most temperate bacteriophage, the Mx8 attP site is contained within the int gene and upon insertion into the M. xanthus chromosome, the 3′ end of the int gene is altered. This modified int gene produces a protein, IntX, with lower specific integrase activity (8).

Because no natural replicating plasmids have been identified for M. xanthus, or for any other myxobacteria, phage attachment sites provide an efficient and stable alternative for introducing new genes or adding additional copies of existing ones into the cell. The Mx8 int and attachment site can be used to integrate DNA into the chromosome, but expression of many genes is affected by insertion into the Mx8 attB sites; many developmental as well as two constitutive promoters, mgl and pilA, have reduced activity at this site (2, 6). There remains a need for more effective and reliable transformation systems that will enable insertion of DNA into the chromosome of M. xanthus and other bacteria. The present invention meets these and other needs.

### SUMMARY OF THE INVENTION

The present invention provides methods and materials for transforming host cells using a bacteriophage Mx9 transformation system. In another aspect, the present methods, materials, host cells and vectors are directed to enhancing the production of a useful compound, including but not limited to a polyketide, through the introduction of one or more genes into the DNA of a variety of bacterial host cells.

In one aspect, the invention provides a method for modification of a DNA of a bacterial cell comprising in its genome a first attachment site recognized by a protein with Mx9 integrase activity, comprising introducing a Mx9 transformation system into the cell, said system comprising (a) a gene encoding a protein with Mx9 integrase activity protein operably linked to a promoter active in the host cell, and (b) a DNA vector comprising a second attachment site recognized by the integrase protein, which may be the same as the first attachment site.

These and other embodiments of the invention are described in more detail in the following description, examples, and claims set forth below.

### BRIEF DESCRIPTION OF THE FIGURES

FIG. 1 presents a physical map of the int region from Mx9. Boxes represent putative open reading frames. The hatched box in int designates the position of attP.

FIG. 2 presents the nucleotide sequence of the Mx9 int gene [SEQ ID NO:1] and the deduced amino acid sequence [SEQ ID NO:2]. Amino acids are in one-letter code underneath the DNA sequence. The sequence in bold [SEQ ID NO:5] is the Mx9 attP core site. Arrows represent inverted repeats. A previous version of this sequence had the following differences: 504 A-->T and 505 G-->A.

FIG. 3 presents (A) Nucleotide sequence of the Mx9 attB1 site [SEQ ID NO:3] and (B) Nucleotide sequence of the Mx9 attB2 site [SEQ ID NO:4]. Nucleotides in bold are the 42 bp [SEQ ID NO:5] identical in the Mx9 attP site. Underlined nucleotides encode tRNA^{gly}. Arrows; inverted repeat within attB2. (C) Nucleotide sequence of the native Mx9 attB1 [SEQ ID NO:6]. Nucleotides in bold indicate the partial core sequence. (D) Nucleotide sequence of the attP site [SEQ ID NO:7]. Arrows; inverted repeat.

FIG. 4 presents the predicted cloverleaf secondary structure for tRNAgly from M. xanthus [bases 1397 to 1428 of SEQ ID NO:1]. The bases that are contained within the core attB sequence are outlined.

FIG. 5 shows an agarose gel of PCR amplified DNA fragments. Lanes 1. 100 bp ladder from New England Biolabs. Lane 2. PCR amplification reactions for detection of attB2 in the wild type strain DZ1. Lanes 3 and 4. PCR amplification reactions for detection of attB2 in two independent isolates that contain a plasmid integrated at attB1. Lanes 5 and 6. PCR amplification reactions for detection of attB2 in two independent isolates that contain a plasmid integrated at attB2.

FIG. 6A shows the lacZ gene transcribed from the pilA promoter integrated at the either the pilA chromosomal location, Mx9 attB1 or attB2, or the Mx8 attB sites. FIG. 6B and FIG. 6C show the lacZ gene transcribed from the mgl promoter integrated at the either the mgl chromosomal location, Mx9 attB1 or attB2, or the Mx9 attB sites.

FIG. 7 shows the consensus sequence of a Chrysoperla carnea transposase gene [SEQ ID NO:19].

### DETAILED DESCRIPTION OF THE INVENTION

The present invention provides methods and materials for transforming bacterial cells using a bacteriophage Mx9 transformation system (also called an Mx9 enzyme system). In one aspect, the invention provides an Mx9 transformation system that may be used to introduce DNA into a bacterial cell comprising an attB site. The Mx9 transformation system comprises (1) a gene encoding a protein with Mx9 integrase activity and (2) a DNA vector comprising an attachment site (attP) recognized by the attachment site. The int gene product catalyses recombination between the attP and attB sites, resulting in integration of DNA sequences from the DNA vector. Proteins with Mx9 integrase activity, the attP site, and attB site are described in detail below.

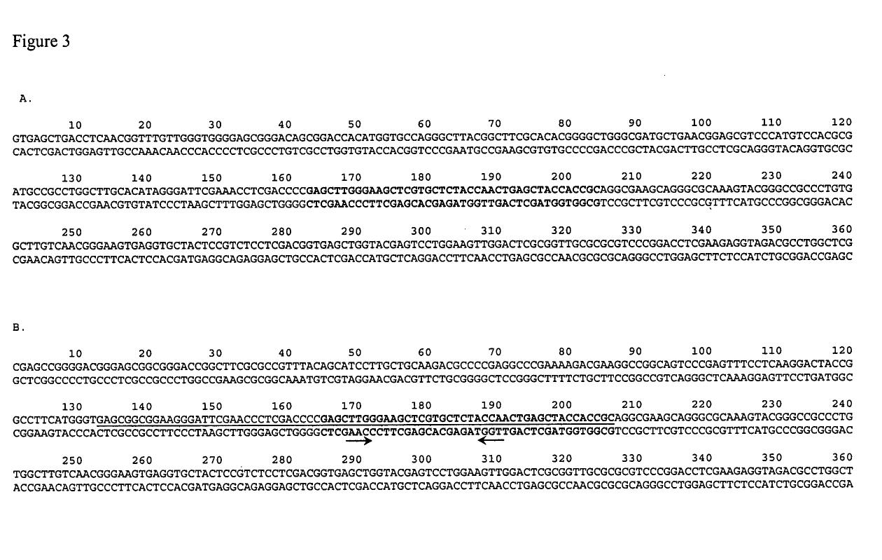

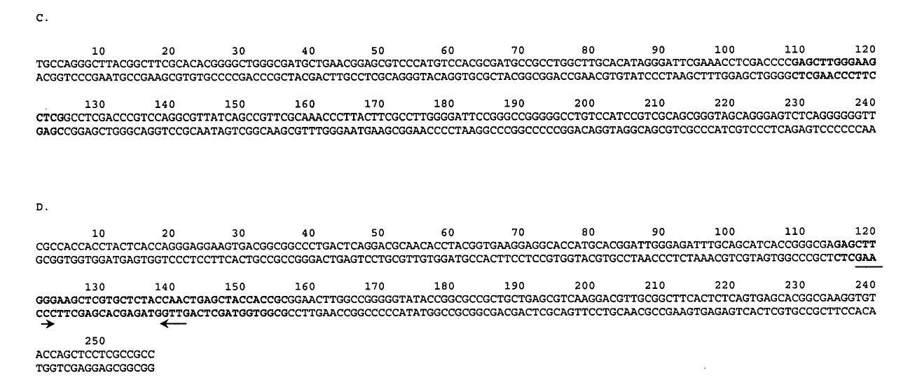

In one embodiment of the invention, the attB site comprises the 42-b core sequence [SEQ ID NO:5]. The attB site may further include at least a portion of the sequences flanking the attB1 and/or attB2 site core sites (e.g., attR and attL, discussed below, which comprise portions of SEQ ID NOS: 3, 4 and 6). In an embodiment, the attP site comprises the 42-b core sequence [SEQ ID NO:5]. The attP site may further include at least a portion of the sequences flanking the core sequence, e.g., as shown in FIG. 3D. In an embodiment, the protein with Mx9 integrase activity (hereinafter, “int protein”) is the product of the int gene having the sequence of SEQ ID NO:2. It will be apparent to the reader that the attB site, attP site and int protein used in the practice of the invention need not be identical to those of the naturally occurring Mx9-Myxococcus xanthus system and that the invention can be practiced using an having sequences substantially identical to those of the naturally occurring sequences. For example, the int protein can differ from SEQ ID NO:2 by conservative amino acid replacements or other substitutions, so long as it has Mx9 integrase activity, i.e. catalyses recombination between attP and attB sites having the sequences of SEQ ID NO:7 and 4, respectively (see FIG. 3). Conversely, the attP and attB sites can differ from naturally occurring sites (and may comprise only a fraction of SEQ ID NO:7, 3, 4, or 6), so long as they are recognized by the int protein having a sequence of SEQ ID NO:2.

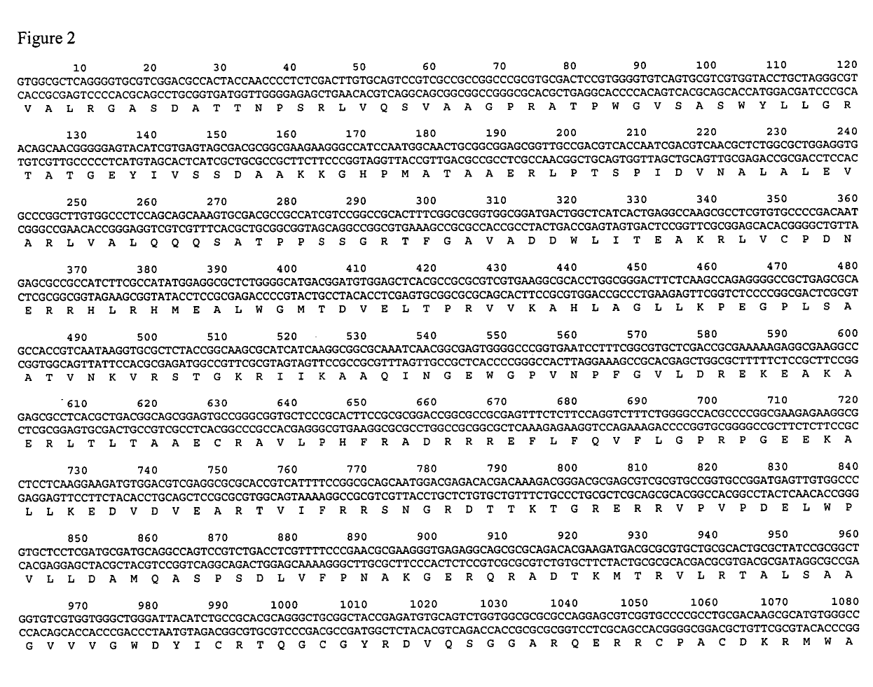

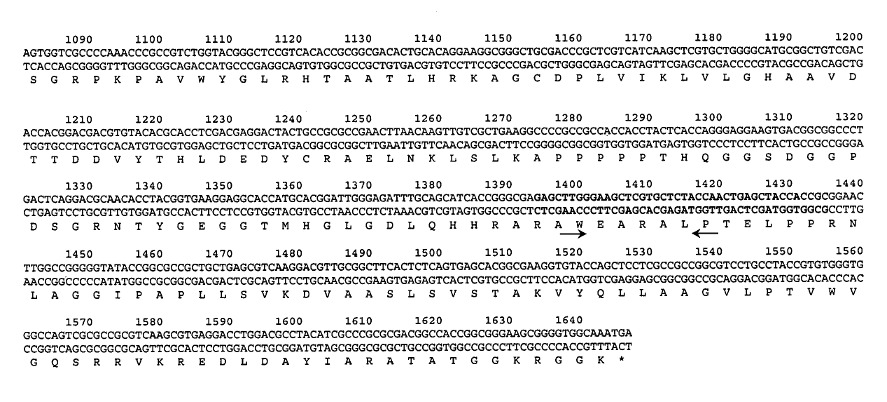

In one embodiment, the protein with Mx9 integrase activity has the sequence shown in FIG. 2 [SEQ ID NO:2], or has a substantially identical sequence. In this context, substantial sequence identity means at least about 70%, more often at least about 80%, most often at least about 90% identity. Sequence identity can be calculated according to the method of Pearson and Lipman, 1988, Proc. Natl. Acad. Sci. U.S.A. 85:2444 using default parameters. In an aspect the invention provides an integrase having the sequence shown in FIG. 2 [SEQ ID NO:2] or having a substantially identical sequence and having integrase activity (e.g., when substrates are the sequence of attP and attB2 sites shown in FIG. 3). In an aspect, the integrase is encoded by a DNA having the sequence of SEQ ID NO:1 or a substantially identical sequence, e.g., at least about 70%, at least about 80%, at least about 90%, or at least about 95% identical (which can be calculated for nucleic acids using the method of Altschul, 1990, J. Mol. Biol. 215:403-10 using default parameters). In another aspect, the invention provides an isolated or recombinant DNA molecule comprising the sequence of SEQ ID NO:1 or a substantially identical sequence (e.g., at least about 70%, more often at least about 80%, most often at least about 90% identity). In a related aspect, the invention provides an isolated or recombinant DNA molecule comprising a sequence encoding SEQ ID NO:2 or a substantially identical sequence (e.g., at least about 70%, at least about 80%, or at least about 90% identity). In some embodiments the isolated or recombinant DNA is less than 5000, less than 1000, less than 5000 or less than 2000 bases in length. In one aspect, the invention provides a recombinant vector comprising an integrase encoding gene. In an embodiment, the gene is operably linked to a promoter that functions in a host cell, so that upon introduction into a cell the integrase is expressed in a host cell.

In an aspect, the attP and attB sites comprise the 42-base core sequence, and may also comprise at least about 10, at least about 20, at least about 30, at least about 40, at least about 50, at least about 100, or all, of one or more of the flanking sequences shown for attP, attB1 or attB2 in FIG. 3 [e.g., SEQ ID NOS:7, 3, and 4 respectively], or a substantially identical sequence. The attB and attP core sequences may be sufficient for recombination. Alternatively, at least a portion of the flanking sequence(s) may be necessary for recombination or improve recombination frequency. The precise extent of sequence required for efficient recombination can easily be determined using routine assays for recombination using a series of constructs comprising different amounts of sequence.

In an aspect, the invention provides an isolated or recombinant DNA molecule comprising a sequence selected from a sequence comprising the Mx9 attB1 site [SEQ ID NO:3]; the Mx9 attB2 site [SEQ ID NO:4]; the Mx9 native attB1 site [SEQ ID NO:6], the attR site of attB1 [nucleotides 205-360 of SEQ ID NO:3], the attR site of attB2 [nucleotides 207-360 of SEQ ID NO:4], the attL site of attB1 [nucleotides 1-162 of SEQ ID NO:3] or the attL site of attB2 [nucleotides 1-164 of SEQ ID NO:4], or, alternatively, at least about 10, at least about 20, at least about 30, at least about 40, at least about 50, at least about 100, from, or all of, an aforementioned sequence. In some embodiments the isolated or recombinant DNA is less than 5000, less than 1000, less than 500 or less than 200 bases in length. In an aspect, the invention provides an isolated or recombinant DNA molecule comprising a 42 base sequence corresponding to nucleotides 165-206 of SEQ ID NO:4, i.e., SEQ ID NO:5. In an aspect, the invention provides an isolated or recombinant DNA molecule comprising an attP sequence. In one embodiment the attP sequence consists of or comprises SEQ ID NO:5, or alternatively, SEQ ID NO:7, or at least 50, at least 100, or at least 150 bases of SEQ ID NO:7 (generally including the core sequence). The invention provides recombinant vectors comprising any of the aforementioned DNA molecules.

In one aspect the attB and attP sites comprise identical sequences, e.g., 42 base pair core sequences. In an embodiment, the attB site is located within the 5′ region of the tRNA^{gly} gene of the host cell. In another aspect, the one or more attB sites are comprised of attB1 and/or attB2. In an embodiment, the present invention provides methods wherein the target DNA for the Mx9 transformation system comprises flanking sites attR and attL, and the integrase protein, when expressed, is an enzyme that facilitates site-specific recombination through binding to the attP and attB sites.

The int gene and attP site may be situated on the same vector. However, the integrase can function in trans and, accordingly, the sites can be introduced on different vectors. In another embodiment of the invention, the vector comprising an attP site is introduced into a recombinant cell expressing the int gene (e.g., a cell stably transformed with int protein encoding gene). As used herein, “vector” has its usual meaning in the art, and refers to polynucleotide elements that are used to introduce recombinant nucleic acid into cells for either expression or replication. Exemplary vector classes include recombinant DNA or RNA constructs, such as a plasmid, a phage, recombinant virus or other vectors. An “expression vector” is a vector capable of expressing DNAs that are operatively linked with regulatory sequences, such as promoter regions. It will be appreciated by those of skill that the vectors may contain additional elements for selection (e.g., antibiotic resistance markers), cloning (e.g., polylinkers), replication, and the like. Appropriate expression vectors are well known to those of skill in the art and include those that are replicable in prokaryotic cells, and those that remain episomal or those which integrate into the host cell genome (the term “host” cell refers to the cell into which the attP containing vector is introduced). It will be appreciated that a naturally occurring (non-recombinant) Mx9 phage is not itself a vector, although a recombinant Mx9 phage modified to carry a heterologous DNA would be considered a vector.

The integrase gene of the Mx9 transformation system is operably linked to a promoter that functions in the intended host. Numerous prokaryotic, viral and synthetic promoters are known in the art and include, for example act promoters, tcm promoters, promoters derived from sugar metabolizing enzymes, such as galactose, lactose (lac) and maltose, promoters derived from biosynthetic enzymes such as for tryptophan (trp), the β-lactamase (bla), bacteriophage lambda PL and T5, synthetic promoters, such as the tac promoter (U.S. Pat. No. 4,551,433), and mariner-type promoters may be used Exemplary promoters for Myxococcus cells include the native int gene promoter, the pilA promoter and the mgl promoter (see Wu and Kaiser, 1997, “Regulation of expression of the pilA gene in Myxococcus xanthus” J. Bacteriol. 179:7748-7758 and GenBank accession number AF377950).

The methods of the present invention may be used to transform any of a variety of host cells that comprise an attB attachment site recognized by the int gene product. Importantly, cells that lack a required integration or attachment site can be genetically engineered to contain one or more such sites, and the integrase gene can be placed under the control of a desired promoter. Thus, the invention can be applied to virtually any host cell. The invention is particularly suited for Myxobacteria, such as Sorangium or Myxococcus. In certain embodiments, the host cells of the present invention may be Sorangium cells (e.g., Sorangium cellulosum), Myxococcus cells (e.g., Myxococcus xanthus), Cystobactera, bacteria of order Stigmatella (e.g., S. erecta and S. aurantiaca), Pseudomonas cells, or Streptomyces cells.

Methods for introducing the recombinant vectors and exogenous DNA molecules of the present invention into suitable hosts are known to those of skill in the art and typically include the use of CaCl₂ or other agents, such as divalent cations, lipofection, DMSO, protoplast transformation, conjugation, or electroporation. References herein to “ransformation” and its grammatical equivalents is intended to encompass any method of introducing an exogenous DNA into a cell.

In one aspect, the present invention is directed to methods of transforming deoxyribonucleic acid (DNA) into a bacterial host cell to effectuate or improve polyketide expression. In one embodiment, the method comprises a) introducing a gene to the DNA of a bacteriophage Mx9 transformation system, said system comprising a gene encoding an integrase protein (int) and an attachment site (attP); b) introducing said bacteriophage Mx9 transformation system to a host cell that contains a nucleotide sequence encoding a polyketide and one or more integration sites (attB) located in the DNA of said host cell; and c) transforming said host cell with said gene by site-specific recombination at the one or more attB sites.

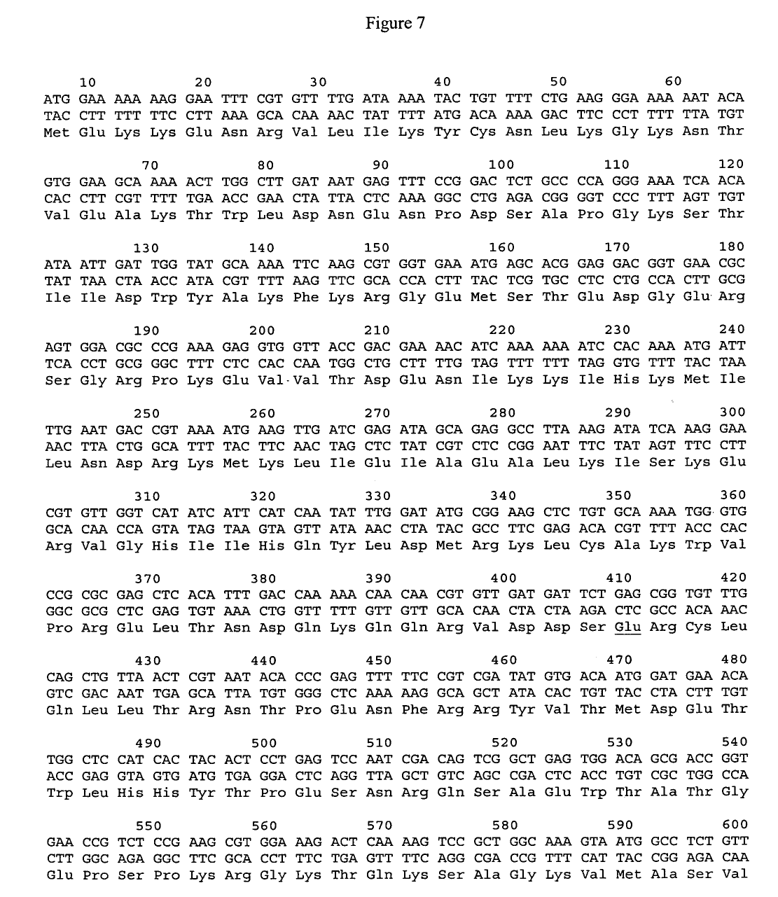

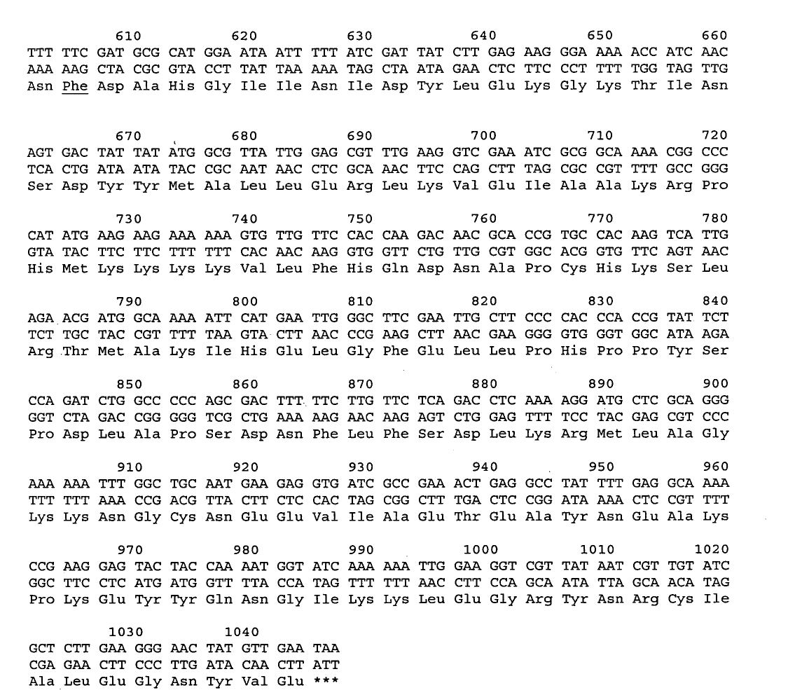

As noted, the invention provides materials and methods useful for insertion of a gene or genes into a host cell, even if that host cell lacks an Mx9 attachment site. Thus, in accordance with the methods of the invention, such host cells can be modified to include the required attachment site. One useful method for modifying host cells to include an Mx9 attachment site is transposon-based transformation (see provisional patent application No. 60/403,290 (filed Aug. 13, 2002) and U.S. patent application Ser. No. 10/_______, filed Aug. 13, 2003, entitled “Transposon-Based Transformation System,” having attorney docket number 30062-2009800). In one embodiment, a transposon vector comprising (1) inverted terminal repeat sequences (ITRs) comprising the sequence ACAGGTTGGCTGATAAGTCCCCGGTCT [SEQ ID NO:17] GGATCCAGACCGGGGACTTATCAGCCAACCTGT [SEQ ID NO:18] and (2) a gene encoding a transposase having a sequence shown in FIG. 7, optionally comprising an E137K mutation, operably linked to a T7A1 promoter (Lanzer et al., 1988, Proc. Nat'l Acad Sci 85:8973-77) is used. In one embodiment, an attB site is introduced into a bacterial cell genome by a) transforming the cell with a transposon vector comprising inverted repeat sequences and a nucleotide sequence comprising a bacteriophage Mx9 integration site (attB), whereby the transposon vector transposes into the DNA of said cell; b) introducing a gene to the bacteriophage Mx9 transformation system, said system comprising a gene encoding an integrase protein (int) and an attachment site (attP); c) introducing said bacteriophage Mx9 transformation system to a host cell; and d) transforming said host cell with said gene by site-specific recombination at said attB site. In one aspect, the invention provides a method for a) transforming a cell that contains a nucleotide sequence encoding a polyketide synthase with a transposon vector comprising inverted repeat sequences and a nucleotide sequence comprising a bacteriophage Mx9 integration site (attB), whereby the transposon vector transposes into the DNA of said cell; b) introducing a gene into a bacteriophage Mx9 transformation system, said system comprising a gene encoding an integrase protein (int) and an attachment site (attP); c) introducing said bacteriophage Mx9 transformation system to a host cell; and d) transforming said host cell with said gene by site-specific recombination at said attB site.

In another aspect, vectors useful for introducing genes into host cells containing an Mx9 integration site are provided. In a particular aspect, vectors of the present disclosure include (1) vectors (including bacteriophage and plasmid vectors) comprising DNA encoding an Mx9 phage attachment site (attP), and another gene, and (2) vectors comprising DNA encoding an integrase protein, an Mx9 phage attachment site (attP), and another gene. The other gene can be any DNA sequence that is desired to be introduced into the target cell, whether encoding a protein or not. As described below, in some embodiments, the gene changes or improves polyketide production in a polyketide producing cell.

In another aspect, the present invention provides host cells, including e.g., M. xanthus host cells, comprising genes introduced by the described methods. In one embodiment, the present methods, materials, host cells and vectors are directed to enhancing the production of a useful compound, including but not limited to a polyketide, through the introduction of one or more genes into the DNA of a variety of bacterial host cells. Thus, in one aspect, transformed host cells are provided that are produced by the claimed methods, which host cells comprise one or more genes integrated to effectuate or improve polyketide expression by the cell. For example, M. xanthus may be used, for example, for the production of epothilone (4; U.S. Pat. No. 6,410,301 “Myxococcus host cells for the production of epothilones”) and genes may be introduced into such epothilone-producing cells to affect the amount, structure or other characteristics of the polyketide produced. In one embodiment, host cells of the present invention are epothilone-producing cells, wherein the epothilone produced is generally selected from epothilone A, B, C, and D.

In one aspect, a gene that improves polyketide production upon functional integration into the DNA of a host cell is introduced into a cell that expresses, or can be engineered to express, a polyketide synthase. In one aspect, the genes introduced into a host cell by the methods of the invention comprise an operon of aprpE gene, accA, and pccB genes to produce increased quantities of malonyl-CoA and/or methylmalonyl-CoA. The genes can be under the control of a suitable promoter, such as a PKS promoter, i.e., from epothilone (U.S. Pat. No. 6,303,342; U.S. patent application Ser. No. 09/957,483, filed Sep. 19, 2001), soraphen (U.S. Pat. No. 5,716,849, incorporated herein by reference), or tombamycin (U.S. patent application Ser. No. 09/942,025, filed Aug. 28^{th}, 2001, and U.S. Pat. Nos. 6,280,999, and 6,090,601, each of which is incorporated herein by reference) gene clusters. The gene or genes are inserted in a recombinant bacteriophage Mx9 of the invention and then integrated into the DNA of the host cell. In one aspect the prpE gene, accA, and pccB genes are inserted into a Myxococcus xanthus cell.

In another aspect, the genes inserted into the host cell may comprise a matB gene or an operon comprising matB and matC genes, such as those from Rhizobium leguminosarum bv. trifolii, which respectively encode a ligase that can attach a CoA group to malonic or methylmalonic acid and a transporter molecule to transport malonic or methylmalonic acid into the host cell respectively, to produce increased quantities of malonyl-CoA and methylmalonyl-CoA (U.S. patent application Ser. Nos. 09/687,555, filed Oct. 13, 2000; 09/798,033, filed Feb. 28, 2001; and 10/087,451, filed Feb. 28, 2002; each of which is incorporated herein by reference).

In another aspect, vectors useful for introducing genes into host cells containing an Mx9 integration site are provided. In a particular aspect, vectors of the present disclosure include bacteriophage vectors comprising DNA encoding an integrase protein, an Mx9 phage attachment site (attP), and another gene. In an embodiment, the vector is a plasmid vector. In a related aspect, the invention provides a vector selected from the group consisting of pKOS35-93, pKOS35-117.9.7, pKOS249-12, pKOS249-23, and pKOS249-31. In one aspect of the invention, an Mx9 transformation system is used to introduce DNA into a host chromosome.

In related aspects, the invention provides a method of transforming a bacterial host cell, said method comprising the steps of a) introducing a first gene into a bacteriophage Mx9 transformation system, said system comprising a second gene encoding an integrase protein (int) and an attachment site (attP); b) introducing said bacteriophage Mx9 transformation system to a host cell that contains one or more integration sites (attB) located in the DNA of said host cell; and c) transforming said host cell with said first gene by site-specific recombination at the one or more attB sites. In an embodiment, the one or more attB sites are comprised of attB1 (SEQ ID NO:3), attB2 (SEQ ID NO:4), or a combination thereof. In an embodiment, the cells are Myxococcus cells, for example epothilone-producing cells. In an embodiment, the epothilone is selected from the group consisting of epothilone C and D. In some embodiments, the first gene is selected from the group consisting of prpE, accA, pccB, matB and matC genes. In an embodiment of the invention, the attB and attP sites are comprised of identical sequences, which may be identical 42 base pair sequences corresponding to nucleotides 1394-1435 of SEQ ID NO:1. In an embodiment, the attB site is located within the 5′ region of the tRNA^{gly} gene. In an embodiment of the method, DNA from said attR site is deleted upon transformation of said host cell. In an embodiment, the gene encoding an integrase protein is altered upon transformation of said host cell.

The invention also provides a transformed bacterial host cell produced by an aforementioned method. In an embodiment, the host cell produces an epothilone selected from epothilone A, B, C, and D. Optionally, the first gene is selected from the group consisting of prpE, accA, pccB, matB and matC genes.

In an aspect, the invention provides a method of transforming a bacterial host cell that lacks a bacteriophage Mx9 integration site (attB) to improve polyketide expression, said method by a) transforming a host cell with a transposon vector comprising inverted repeat sequences and a nucleotide sequence comprising a bacteriophage Mx9 integration site (attB), whereby the transposon vector transposes into the DNA of said cell; b) introducing a first gene to a bacteriophage Mx9 transformation system, said system comprising a second gene encoding an integrase protein (int) and an attachment site (attP); c) introducing said bacteriophage Mx9 transformation system to the host cell; and d) transforming said host cell with said first gene by site-specific recombination at said attB site. According to this method, the host cells may be Sorangium cells, Myxococcus cells, Pseudomonas cells, or Streptomyces cells as well as others. In embodiments, the host cells produce epothilone selected from epothilone A, B, C, and D and/or the first gene is selected from the group consisting of prpE, accA, pccB, matB and matC genes and/or the attB site comprises flanking sites attR and attL, and said integrase protein, when expressed, is an enzyme that facilitates said site-specific recombination through binding to attB and attP sites. The invention further provides a transformed bacterial host cell produced by this method, which optionally may produce an epothilone selected from epothilone A, B, C, and D.

The invention also provides a bacteriophage Mx9 vector comprising DNA encoding an integrase protein, an Mx9 phage attachment site (attP), and another gene.

Experimental Results and Discussion

Materials and Methods

Bacteria, Phage, and plasmids. DZ1 is a nonmotile strain of M. xanthus and was used for plating Mx9 and for characterization of the Mx9 attachment sites (12). DK816 is the natural M. xanthus isolate lysogenic for Mx9 (9). M. xanthus strains were grown in CYE medium (1) or 1% CTS (1% casitone, 0.2% MgSO₄.7H₂O, 50 mM HEPES pH 7.6). Phleomycin (Cayla) was used at a concentration of 30 μg/ml. The Mx9 phage was reisolated from DK816 by growing a culture to stationary phase, pelleting the cells, and plating dilutions of the supernatant onto DZ1. High titer stocks of Mx9 were made by coring a plaque and placing it in phage buffer (10 mM MOPS [pH7.6], 4 mM MgCl₂, 2 mM CaCl₂). The eluted phage were diluted and mixed with 0.5 ml of DZ1 in early stationary phase. After incubating the cells and phage at room temperature for 20 minutes, 2.5 ml of top agar was added and the suspension was poured onto phage plates (1% BBL trypticase, 0.1% MgSO₄.7H₂O, 1% agar, 10 mM MOPS pH 7.6). The plates that gave confluent lysis after 2 days of incubation at 30° C. were overlayed with 5 ml of phage buffer and incubated at 4° C. overnight. The eluted phage were stored at 4° C. Phage stocks greater than 1×10⁹ pfu/ml were obtained with this method. Plasmids used are described in Table 1.

TABLE 1

| Plasmid | Characteristics |
|---|---|
| pKOS35-117.9.9 | amp^{r} kan^{r} colEI, 4.6 kb fragment from M × 9 |
| pKOS139-29 | amp^{r}, colEI, P_{T7A1} M × 8 int attP⁻ |
| pKOS139-47 | tc^{r}, p15A, P_{ingl} lacZ, M × 8 attP |
| pKOS178-86 | tc^{r}, p15A, P_{pilA} lacZ, M × 8 attP |
| pKOS178-177 | tc^{r}, p15A, P_{pilA} lacZ, M × 9 int attP |
| pKOS178-188 | tc^{r}, p15A, P_{ingl} lacZ, M × 9 int attP |
| pKOS249-31 | amp^{r} bleo^{r} colEI, P_{T7A1} M × 9 int attP |

Isolation of phage DNA. The phage from a high titer stock were pelleted by centrifuging in an SS-34 rotor at 28,000 rpm for 3 hours and then resuspended in TE (10 mM Tris [pH7.6] 1 mM EDTA). The phage proteins were removed by extracting twice with phenol and twice with phenol/chloroform/isoamylalcohol. The DNA was precipitated and resuspended in TE.

Isolation and sequence of the phage attachment site. To isolate the phage attachment site, phage DNA was partially cleaved with HinPI and the fragments were ligated into pKOS35-93 cleaved with AccI. The plasmid pKOS35-93 is pBluescriptII SK+ with the kanamycin resistance from Tn5 ligated into the SmaI and EcoRI sites. One plasmid, pKOS35-117.9.7, integrated efficiently into the chromosome. The insert from this plasmid was sequenced

Isolation of the bacterial attachment site. The bacterial attachment site (attB) was isolated by electroporating pKOS35-117.9.7 into DZ1, making chromosomal DNA, and then recovering the plasmid with flanking chromosomal DNA. Six kanamycin resistant colonies were picked and chromosomal DNA was prepared from each. The DNA was cleaved with either PstI or XhoI, ligated, and then transformed into E. coli. Three colonies from each of the electroporations were picked and the recovered plasmids were cleaved with PstI or XhoI. One plasmid from each was sequenced using either primer 183-66.3 (GAAGGAGGCACCATGCACGG [SEQ ID NO:8] or 183-66.4 (CTCACTGAGAGTGAAGCCGC [SEQ ID NO:9]).

PCR amplification of the Mx9 attB. Primers were designed to PCR amplify attB1 and attB2. Primers 183-99.4 (CGAGGTCCGGGACGCGCGCA [SEQ ID NO:10]) and 183-99.6 (TGCCAGGGCTTACGGCTTC [SEQ ID NO:11]) were used to amplify a 285 bp attB1 fragment and 183-99.5 (TATCCCAGCAACCGCCGGAG [SEQ ID NO:13]) with primer 183-99.4 was used to amplify a 373 bp attB2 fragment. To amplify the native attB1 site primers 183-99.6 and 249-179.7 (CAGCACGGGTGCAGCAAC [SEQ ID NO:14]) were used to amplify a 250 bp fragment. PCR reactions were done using chromosomal DNA from DZ1 and the FailSafe™ PCR system from Epicentre. Amplification conditions were 96° C. for two minutes and then 30 cycles of 94° C. 30 seconds, 55° C. for 1 minute, 72° C. for 2 minutes.

Construction of a minimal integration plasmid. The int gene was PCR amplified from pKOS35-117.9.7 using the primers 111-74.4 (CCCAATTGGCTCAGGGCAGCGGCTCATT [SEQ ID NO:15]) and 111-82.5 (CCCCATGGCGCTCAGGGGTGCGTCGGACGCC [SEQ ID NO:16]). PCR amplification conditions were those previously described. The amplified fragment was ligated into the EcoRV site of pLitmus 28 (New England Biolabs) to create pKOS249-12. The int gene was removed from this plasmid by cleaving with EcoRI, the DNA ends were made blunt with the Klenow fragment of DNA polymerase followed by cleaving with NcoI. The fragment was ligated with pUHE24-2B (3) that was cleaved with PstI, the DNA ends were made blunt with the Klenow fragment of DNA polymerase I and cleaved with NcoI. The resulting plasmid, pKOS249-23, contains the int gene under the control of the E. coli phage T7 A1 promoter that has been engineered to contain 2 LacI binding sites to repress transcription. The bleomycin resistance gene was added to this plasmid by isolating the bleomycin resistance gene from pKOS183-112 as a BamHI to HindIII fragment, the DNA ends were made blunt with the Klenow fragment of DNA polymerase I and ligating it with pKOS249-23, which was cleaved with XhoI and the DNA ends were made blunt with the Klenow fragment of DNA polymerase I. This plasmid is designated pKOS249-31.

β-galactosidase assays. Seed cultures of two isolates for each integration site were grown in 1% CTS (5 ml) to mid to late log phase. To start the assay cultures, 35 ml of CTS was inoculated with 1 ml of seed culture at an OD₆₀₀ of 0.073. β-galactosidase assays were performed by removing an aliquot of cells and adding them to Z buffer for a combined volume of 1 ml. The cells were lysed by adding one drop of 0.1% SDS, two drops of chloroform, and vortexing the sample for 5 seconds. The assay was initiated by the addition of 0.1 ml of O-nitrophenyl β-D-galactopyranoside (8 mg/ml) and mixing. The reactions were stopped by the addition of 0.5 ml of 1 M Na₂CO₃. The OD₆₀₀ of the cell culture and the OD₄₂₀ of the enzyme reactions were determined using a SpetraMax 250 plate reader. Miller units were determined as previously described (10).

Accession numbers. The Mx9 sequence has been assigned the accession number AY247757. The accession numbers for attB1 and attB2 are AY297770 and AY297771, respectively.

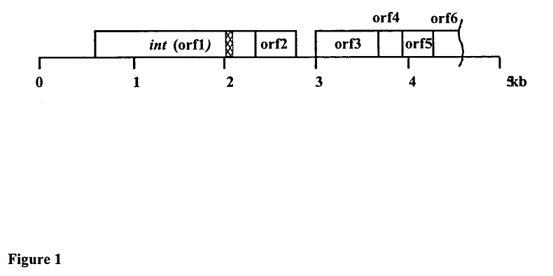

Identification of the Mx9 int and attachment site. To identify the int gene and attachment site, a library of 5-8 kb fragments of Mx9 was made, and a clone that was able to integrate into the M. xanthus chromosome was identified. The insert in this plasmid, pKOS35-117.9.7, was sequenced. Five complete and one partial open reading frames (orf) were identified in the 4.6 kb fragment (FIG. 1). Orf 1 was the only reading frame that showed amino acid similarity with other known integrase genes, and therefore was given the gene designation int. The other orfs resembled orfs from Mx8; orf2, orf3, orf4, orf5, and orf6 showed similarity to P15, P14, P16, P17, and P18, respectively from Mx8. From the degree of similarity of these orfs between, it appears that Mx8 and Mx9 are very similar phages.

The Mx9 int gene was examined for sequences that would indicate an attachment site. Analysis revealed a DNA segment within the int gene (nt 1397-1428 (FIG. 2)) that had sequence similarity to tRNA^{gly} from various organisms. Since Mx8 integrates into the tRNA^{Asp} gene of M. xanthus, the sequence that showed similarity with tRNA^{gly} was predicted to serve as the site of integration for Mx9.

To test this prediction, chromosomal DNA from six integrants containing pKOS35-117.9.7 were cleaved with restriction enzymes, ligated, and transformed into E. coli to recover the plasmid along with flanking chromosomal DNA. Sequencing, using primers adjacent to the proposed attachment site, revealed that the point of recombination was indeed that of the putative tRNA^{gly}. Furthermore, the sequence of flanking chromosomal DNA showed that there were two attB sites. It appeared from the number of integrants at each site, 3 for attB1 and 3 for attB2, that both served equally well as the insertion site (FIG. 3).

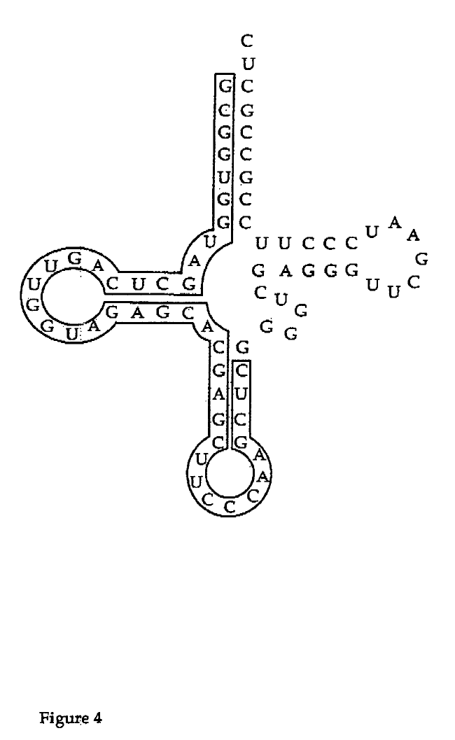

Structure of the two attB sites. FIG. 3 shows 360 bp from each of the attB sites. Both have a common 42 bp core sequence that is also found within the Mx9 int gene. In addition, there are 22 bp 5′ to both attB sites that are identical in 21 positions. There is a putative inverted repeat that may play a role in Integrase protein binding at the attB and attP (FIG. 3b). The site of integration within attB2 lies in the 5′ end of tRNA^{gly} gene, which is underlined in FIG. 3b. However, the sequence of attB1 does not contain a complete tRNA^{gly} gene. FIG. 4 shows the predicted folding of this segment of attB2 into a corresponding tRNA.

Analysis of the attR and attL half-sequences for both attB sites reveals the two attR are identical whereas the attL differ. This is also the case with the two Mx8 attB sites (7). Plasmids containing the Mx8 int gene preferentially integrate at attB1, and this integration often is accompanied by a deletion between attB1 and attB2 (8).

To determine if the identical attR sites are due to the presence of two attB sites containing with identical attR sites or due to the deletion of the DNA between the two attB sites after integration into one of them, PCR analysis was performed using either primer pair 183-99.4 and 183-99.6 for attB1 or 183-99.4 and 183-99.5 for attB2.

A PCR fragment was detected using primers specific for attB2 but none was detected using primers specific for attB1 (data not shown). This suggests that a deletion may occur upon integration of attB1 but to be certain that the lack of a PCR product was not due to the failure to PCR amplify the DNA fragment, further experiments were performed.

Next, the genomic sequence of M. xanthus strain DK1622, generated by Monsanto and available at the TIGR web site, was examined for the two attB sites (www.TIGR.org). The attB2 sequence was almost identical to that previously identified (FIG. 3B) but only the first 178 bp of the attB1 site from FIG. 3A was present before the sequence diverged. Using this sequence information for attB1, a primer was designed that was approximately 100 bp downstream from the point at which the sequence diverged (249-179.7). Using this primer along with 183-99.6, the one 5′ to the attB1 site, and DZ1 genomic DNA, a PCR product of approximately 250 bp was isolated and sequenced. The PCR product was identical to that obtained from the DK1622 genomic sequence (FIG. 3C). Analysis of this sequence reveals that only 16 bp of the 42 bp core att site are present in the native attB1 site.

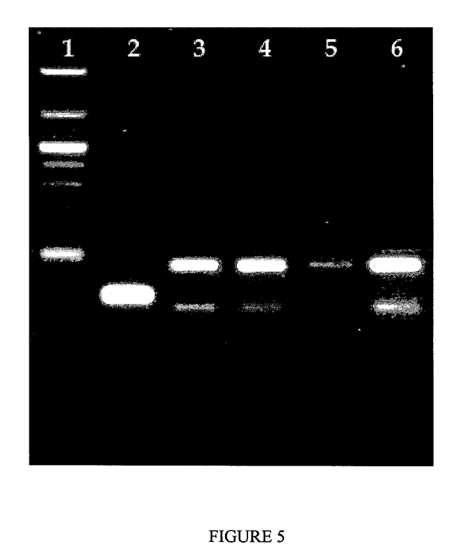

Final proof that a deletion does occur between attB1 and attB2 is shown in FIG. 5. Using the primer pair 183-99.4 and 183-99.5, the ones that amplify the attB2 site, PCR amplification was performed using genomic DNA from the wild type strain or strains harboring a plasmid integrated at either attB1 or attB2. Using chromosomal DNA from DZ1, a strain with no plasmids integrated at either attB site, a 372 bp PCR product containing the attB2 site was detected in lane 2 FIG. 5. Two strains that contain insertions at attB2, lanes 5 and 6 (FIG. 5) do not give the 372 bp band and should not amplify the attB2 due to the presence of a plasmid integrated at that site. If a deletion does occur between attB1 and attB2, then there should be no detectable amplification of attB2 when a plasmid integrates at attB1. Lanes 3 and 4 (FIG. 5) shows that no attB2 PCR product is detected, indicating a deletion of DNA between attB1 and attB2 when an integration occurs at attB1.

Integration results in the alteration of the carboxy terminus of the Mx9 Int protein. Because attP lies within the int gene, integration into the chromosome should alter the 3′ end of int gene is altered. From the 1160 bp of attR that has been sequenced, no stop codon has been identified (data not shown). Thus 70 amino acids from Int should be removed and more than 389 amino acids should be added to the Int protein that is synthesized after integration into the chromosome. These additional amino acids presumably will reduce the enzymatic activity of Int because the IntX protein of Mx8 has lost 112 residues and added 13 amino acids, and is a less active at site specific recombination (8).

Mx9 Int is the only phage protein required for integration. To determine whether int is necessary and sufficient for integration, the int gene was PCR amplified and ligated into an E. coli expression vector that uses an engineered phage T7 A1 promoter. The plasmid pKOS249-31, when electroporated into DZ1, integrated efficiently into the chromosome; approximately 1×10⁴ colonies were obtained per microgram of DNA. Thus, the Mx9 int gene is the only phage encoded protein required for integrative recombination into the bacterial chromosome.

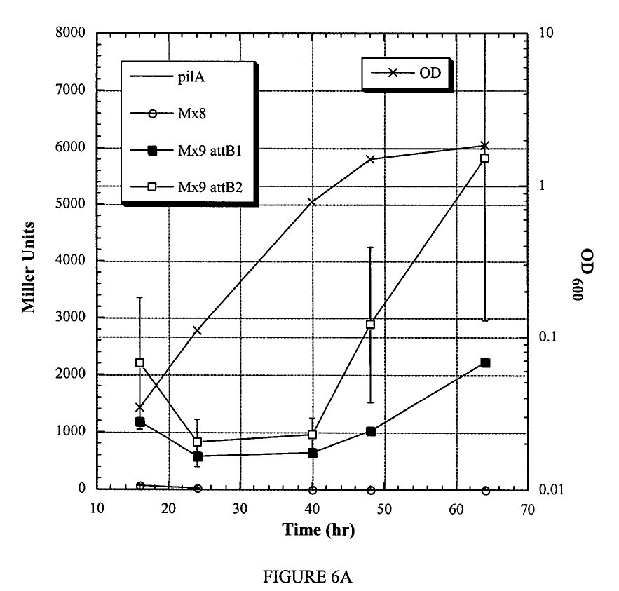

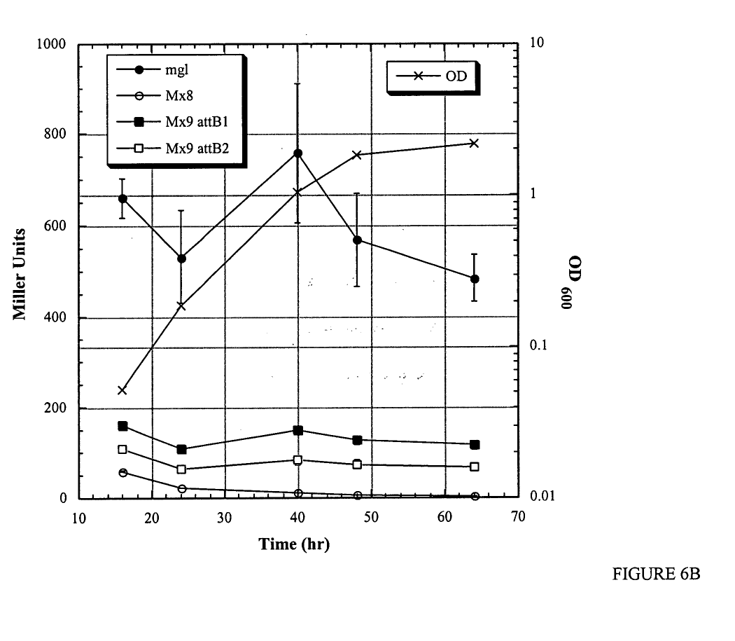

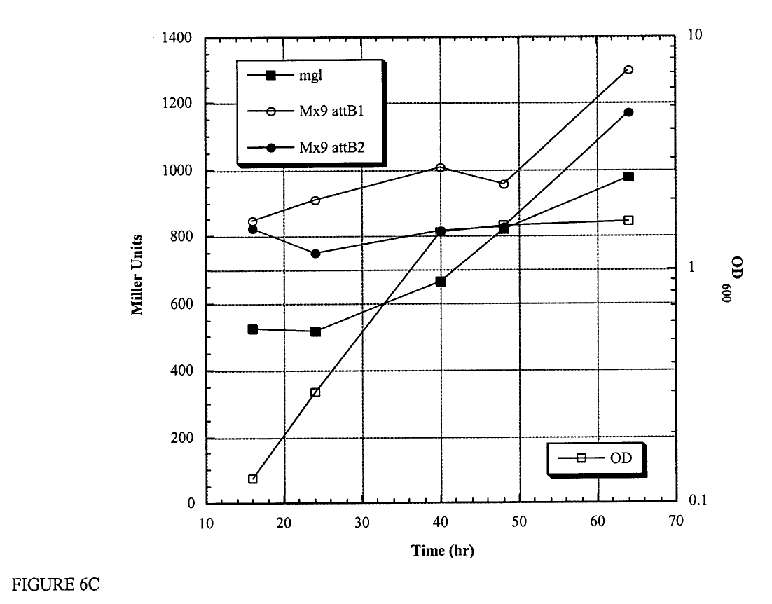

Transcription from the pilA and the mgl promoters integrated at the two Mx9 attB sites. To find a phage attachment site on the M. xanthus chromosome that supported efficient expression of genes from a variety of promoters, fusions of lacZ to the mgl or pilA promoters were constructed and transcription from these promoters at the two Mx9 attB, the Mx8 attB, and the native chromosomal location was analyzed. FIG. 6A shows the expression level of the pilA promoter (P_{pilA}) at the four different locations. Surprisingly, there was little transcription when the P_{pilA} plasmid was integrated by homologous recombination at the pilA location (pKOS178-86). This suggests that there may be a deletion in the pilA promoter region that abolishes activation of the pilA promoter in DZ1 since there was no expression in several isolates that were examined. As we have observed previously, little transcription from P_{pilA} is seen when integrated at Mx8 attB site (pKOS178-86+pKOS139-29). However, the Mx9 sites show high levels of transcription from P_{pilA} (pKOS178-177) and they are fairly similar at both sites, although attB2 had high variability of expression from the two isolates examined. In addition, the regulation at both sites was similar; transcription from P_{pilA} increased during late log and stationary phases.

The results of transcription from the mgl promoter (P_{mgl}) are shown in FIG. 6B. Transcription from P_{mgl} at the two Mx9 attB (pKOS178-188) sites was better than at the Mx8 site (PKOS139-47+pKOS139-29) but not as high when integrated by homologous recombination at the chromosomal mgl. location (PKOS139-47). However, this lower expression at the two Mx9 sites may be vector dependent. Using a plasmid that contained only the attP site and integrating it by supplying the int gene in trans, P_{mgl} functions just as well at both Mx9 sites as it does at the chromosomal mgl location (see FIG. 6C). In this experiment, a plasmid was constructed that contained the mgl promoter fused to lacZ and harbored only the Mx9 attP site. This plasmid was integrated into the Mx9 attB1 or attB2 by co-electroportating it with a second plasmid that expressed the int gene. \overline{β}-galactosidase assays with cells containing this plasmid reveals that the levels of expression from the mgl promoter is as good, if not better, than the native mgl chromosomal location. Thus expression from the mgl promoter at the Mx9 attB locations may be vector dependent. The conclusion from these studies indicates that the Mx9 attB sites are good for expression of foreign or native genes.

The Mx9 int gene and attachment site have been identified, along with the site of integration into the M. xanthus chromosome. The analysis reveals remarkable similarity to the int gene and attachment site from the myxophage Mx8 (7, 8, 11). Both contain the attP within the int gene and integrate within a tRNA gene. They have two attB sites and it appears that adjacent chromosomal DNA is deleted when integration occurs at one of the sites. For both, Int is the only phage-encoded protein needed for integration.

A difference between the Mx8 and Mx9 phage integration systems is the length of their respective core sequences. The core sequence for Mx8 integration is smaller, composed of 29 bp. The attB2 site has two nucleotides that differ at one end, which may account for the preference of Mx8 for inserting at attB1. The att core region for Mx9 is 42 bp, but of the two integration sites only attB2 contains all 42 bases. The attB1 site contains only 16 bases of the core sequence. The lack of a complete core sequence in attB1 may explain why there is always a deletion between attB1 and attB2 when integration occurs at attB1. The Int protein may bind to the inverted repeat within the 42 bp core. Binding of the λ Int protein to its att sites has been shown (5). Since the attB1 contains half of the inverted repeat, only half of the necessary protein complex can form, but once it has assembled, it may interact with the complementary half of proteins from attB2 to allow for integration. This would result in a looping out of the DNA between attB1 and attB2, and its subsequent loss upon integration of DNA.

In our PCR reactions to detect attB1 with primers 183-99.4 & 183-99.6, the conditions were such that if the distance between attB1 and attB2 was less than 2 kb, then a PCR product should have been detected. Since no product was observed, this suggests that the distance between the two sites is greater than 2 kb. Analysis of the DK1622 sequence shows that the two attB sites are 6.7 kb apart. Partial analysis of this sequence shows a couple open reading frames that have sequence similarity to transposase genes, suggesting the presence of a transposon. The other reading frame that was identified reveals high sequence similarity to proteins of unknown functions. Clearly, the open reading frames encoded in between the two attB sites are not critical for growth under laboratory conditions since strains with integrations at attB1 have no visible growth defects.

References

1. Campos, J. M., and D. R. Zusman. 1975. Regulation of development in Myxococcus xanthus: effect of 3′:5′-cyclic AMP, ADP, and nutrition. Proc. Natl. Acad. Sci. U S A 72:518-22.

2. Fisseha, M., M. Gloudemans, R. E. Gill, and L. Kroos. 1996. Characterization of the regulatory region of a cell interaction-dependent gene in Myxococcus xanthus. J Bacteriol 178:2539-50.

3. Julien, B., and R. Calendar. 1995. Purification and characterization of the bacteriophage P4 delta protein. J Bacteriol 177:3743-51.

4. Julien, B., and S. Shah. 2002. Heterologous expression of the epothilone biosynthetic genes in Myxococcus xanthus. Antimicrobial Agents Chemotherapy 46:2772-2778.

5. Landy, A. 1989. Dynamic, structural, and regulatory aspects of lambda site-specific recombination. Annual Review of Biochemistry 58:913-949.

6. Li, S. F., and L. J. Shinikets. 1988. Site-specific integration and expression of a developmental promoter in Myxococcus xanthus. J Bacteriol 170:5552-6.

7. Magrini, V., C. Creighton, and P. Youderian. 1999. Site-specific recombination of temperate Myxococcus xanthus phage Mx8: genetic elements required for integration. J Bacteriol 181:4050-61.

8. Magrini, V., M. L. Storms, and P. Youderian. 1999. Site-specific recombination of temperate Myxococcus xanthus phage Mx8: regulation of integrase activity by reversible, covalent modification. J Bacteriol 181:4062-70.

9. Martin, S., E. Sodergren, T. Masuda, and D. Kaiser. 1978. Systematic isolation of transducing phages for Myxococcus xanthus. Virology 88:44-53.

10. Miller, J. H. 1992. A Short Course in Bacterial Genetics: A Laboratory Manual and Handbook for Escherichia coli and Related Bacteria. Cold Spring Harbor Laboratory Press, NY.

11. Tojo, N., K. Sanmiya, H. Sugawara, S. Inouye, and T. Komano. 1996. Integration of bacteriophage Mx8 into the Myxococcus xanthus chromosome causes a structural alteration at the C-terminal region of the IntP protein. J Bacteriol 178:4004-11.

12. Zusman, D. R., D. M. Krotoski, and M. Cumsky. 1978. Chromosome replication in Myxococcus xanthus. J. Bacteriol. 133:122-129.

Numerous modifications may be made to the foregoing systems without departing from the basic teachings thereof. Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications and patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents.

We claim:

1. A method for modification of a DNA of a bacterial cell comprising in its genome a first attachment site recognized by a protein with Mx9 integrase activity, comprising introducing a Mx9 transformation system into the cell, said system comprising
a) a gene encoding a protein with Mx9 integrase activity protein operably linked to a promoter active in the host cell, and
b) a DNA vector comprising a second attachment site recognized by the integrase protein, which may be the same as the first attachment site.

2. The method of claim 1 wherein the cell is Myxococcus or Sorangium.

3. The method of claim 1 wherein the protein has a sequence at least substantially identical to SEQ ID NO:2.

4. The method of claim 3 wherein the protein has a sequence of SEQ ID NO:2.

5. The method of claim 4 wherein the protein is encoded by a gene comprising the sequence of SEQ ID NO:1.

6. The method of claim 1 wherein said first attachment site comprises SEQ ID NO:5.

7. The method of claim 6 wherein said first attachment site is attB2.

8. The method of claim 1 wherein said second attachment site comprises SEQ ID NO:5.

9. The method of claim 3 wherein said first attachment site has been recombinantly introduced into the cell genome.

10. The method of claim 1 wherein said DNA vector further comprises an exogenous gene.

11. The method of claim 10 wherein the exogenous gene is selected from the group consisting of prpE, accA, pccB, matB, matC and beta-galactosidase genes.

12. The method of claim 6 wherein the first and second attachment sites are comprised of identical sequences.

13. The method of claim 2 wherein the cell is Myxococcus xanthus.

14. The method of claim 13 wherein the cell produces an epothilone.

15. The method of claim 14, wherein the epothilone is selected from the group consisting of epothilone C and D.

16. A bacterial host cell produced by the method of claim 10.

17. The cell of claim 16 wherein that produces an epothilone selected from epothilone A, B, C, and D.

18. The cell of claim 17, wherein said exogenous gene is selected from the group consisting of prpE, accA, pccB, matB and matC genes.


SEQUENCE LISTING (20 sequences)

SEQ ID NO: 1 - DNA, 1647 bp, Bacteriophage MX9
gtggcgctcaggggtgcgtcggacgccactaccaacccctctcgacttgtgcagtccgtcgccgccggcccgcgtgcgactccgtggggtgtcagtgcgtcgtggtacctgctagggcgtacagcaacgggggagtacatcgtgagtagcgacgcggcgaagaagggccatccaatggcaactgcggcggagcggttgccgacgtcaccaatcgacgtcaacgctctggcgctggaggtggcccggcttgtggccctccagcagcaaagtgcgacgccgccatcgtccggccgcactttcggcgcggtggcggatgactggctcatcactgaggccaagcgcctcgtgtgccccgacaatgagcgccgccatcttcgccatatggaggcgctctggggcatgacggatgtggagctcacgccgcgcgtcgtgaaggcgcacctggcgggacttctcaagccagaggggccgctgagcgcagccaccgtcaataaggtgcgctctaccggcaagcgcatcatcaaggcggcgcaaatcaacggcgagtggggcccggtgaatcctttcggcgtgctcgaccgcgaaaaagaggcgaaggccgagcgcctcacgctgacggcagcggagtgccgggcggtgctcccgcacttccgcgcggaccggcgccgcgagtttctcttccaggtctttctggggccacgccccggcgaagagaaggcgctcctcaaggaagatgtggacgtcgaggcgcgcaccgtcattttccggcgcagcaatggacgagacacgacaaagacgggacgcgagcgtcgcgtgccggtgccggatgagttgtggcccgtgctcctcgatgcgatgcaggccagtccgtctgacctcgttttcccgaacgcgaagggtgagaggcagcgcgcagacacgaagatgacgcgcgtgctgcgcactgcgctatccgcggctggtgtcgtggtgggctgggattacatctgccgcacgcagggctgcggctaccgagatgtgcagtctggtggcgcgcgccaggagcgtcggtgccccgcctgcgacaagcgcatgtgggccagtggtcgccccaaacccgccgtctggtacgggctccgtcacaccgcggcgacactgcacaggaaggcgggctgcgacccgctcgtcatcaagctcgtgctggggcatgcggctgtcgacaccacggacgacgtgtacacgcacctcgacgaggactactgccgcgccgaacttaacaagttgtcgctgaaggccccgccgccaccacctactcaccagggaggaagtgacggcggccctgactcaggacgcaacacctacggtgaaggaggcaccatgcacggattgggagatttgcagcatcaccgggcgagagcttgggaagctcgtgctctaccaactgagctaccaccgcggaacttggccgggggtataccggcgccgctgctgagcgtcaaggacgttgcggcttcactctcagtgagcacggcgaaggtgtaccagctcctcgccgccggcgtcctgcctaccgtgtgggtgggccagtcgcgccgcgtcaagcgtgaggacctggacgcctacatcgcccgcgcgacggccaccggcgggaagcggggtggcaaatga

SEQ ID NO: 2 - protein, 548 aa, Bacteriophage MX9
VALRGASDATTNPSRLVQSVAAGPRATPWGVSASWYLLGRTATGEYIVSSDAAKKGHPMATAAERLPTSPIDVNALALEVARLVALQQQSATPPSSGRTFGAVADDWLITEAKRLVCPDNERRHLRHMEALWGMTDVELTPRVVKAHLAGLLKPEGPLSAATVNKVRSTGKRIIKAAQINGEWGPVNPFGVLDREKEAKAERLTLTAAECRAVLPHFRADRRREFLFQVFLGPRPGEEKALLKEDVDVEARTVIFRRSNGRDTTKTGRERRVPVPDELWPVLLDAMQASPSDLVFPNAKGERQRADTKMTRVLRTALSAAGVVVGWDYICRTQGCGYRDVQSGGARQERRCPACDKRMWASGRPKPAVWYGLRHTAATLHRKAGCDPLVIKLVLGHAAVDTTDDVYTHLDEDYCRAELNKLSLKAPPPPPTHQGGSDGGPDSGRNTYGEGGTMHGLGDLQHHRARAWEARALPTELPPRNLAGGIPAPLLSVKDVAASLSVSTAKVYQLLAAGVLPTVWVGQSRRVKREDLDAYIARATATGGKRGGK

SEQ ID NO: 3 - DNA, 360 bp, Bacteriophage MX9
gtgagctgacctcaacggtttgttgggtggggagcgggacagcggaccacatggtgccagggcttacggcttcgcacacggggctgggcgatgctgaacggagcgtcccatgtccacgcgatgccgcctggcttgcacatagggattcgaaacctcgaccccgagcttgggaagctcgtgctctaccaactgagctaccaccgcaggcgaagcagggcgcaaagtacgggccgccctgtggcttgtcaacgggaagtgaggtgctactccgtctcctcgacggtgagctggtacgagtcctggaagttggactcgcggttgcgcgcgtcccggacctcgaagaggtagacgcctggctcg

SEQ ID NO: 4 - DNA, 360 bp, Bacteriophage MX9
cgagccggggacgggagcggcgggaccggcttcgcgccgtttacagcatccttgctgcaagacgccccgaggcccgaaaagacgaaggccggcagtcccgagtttcctcaaggactaccggccttcatgggtgagcggcggaagggattcgaaccctcgaccccgagcttgggaagctcgtgctctaccaactgagctaccaccgcaggcgaagcagggcgcaaagtacgggccgccctgtggcttgtcaacgggaagtgaggtgctactccgtctcctcgacggtgagctggtacgagtcctggaagttggactcgcggttgcgcgcgtcccggacctcgaagaggtagacgcctggct

SEQ ID NO: 5 - DNA, 42 bp, Bacteriophage MX9
gagcttgggaagctcgtgctctaccaactgagctaccaccgc

SEQ ID NO: 6 - DNA, 240 bp, Bacteriophage MX9
tgccagggcttacggcttcgcacacggggctgggcgatgctgaacggagcgtcccatgtccacgcgatgccgcctggcttgcacatagggattcgaaacctcgaccccgagcttgggaagctcggcctcgacccgtccaggcgttatcagccgttcgcaaacccttacttcgccttggggattccgggccgggggcctgtccatccgtcgcagcgggtagcagggagtctcaggggggtt

SEQ ID NO: 7 - DNA, 257 bp, Bacteriophage MX9
cgccaccacctactcaccagggaggaagtgacggcggccctgactcaggacgcaacacctacggtgaaggaggcaccatgcacggattgggagatttgcagcatcaccgggcgagagcttgggaagctcgtgctctaccaactgagctaccaccgcggaacttggccgggggtataccggcgccgctgctgagcgtcaaggacgttgcggcttcactctcagtgagcacggcgaaggtgtaccagctcctcgccgcc

SEQ ID NO: 8 - DNA, 20 bp, Artificial Sequence
  Synthetic Construct
gaaggaggcaccatgcacgg

SEQ ID NO: 9 - DNA, 20 bp, Artificial Sequence
  Synthetic Construct
ctcactgagagtgaagccgc

SEQ ID NO: 10 - DNA, 20 bp, Artificial Sequence
  Synthetic Construct
cgaggtccgggacgcgcgca

SEQ ID NO: 11 - DNA, 19 bp, Artificial Sequence
  Synthetic Construct
tgccagggcttacggcttc

SEQ ID NO: 12 - DNA, 74 bp, Myxococcus xanthus
gcggugguagcucaguugguagagcacgagcuucccaagcucggggucgaggguucgaaucccuuccgccgcuc

SEQ ID NO: 13 - DNA, 20 bp, Artificial Sequence
  Synthetic Construct
tatcccagcaaccgccggag

SEQ ID NO: 14 - DNA, 18 bp, Artificial Sequence
  Synthetic Construct
cagcacgggtgcagcaac

SEQ ID NO: 15 - DNA, 28 bp, Artificial Sequence
  Synthetic Construct
cccaattggctcagggcagcggctcatt

SEQ ID NO: 16 - DNA, 31 bp, Artificial Sequence
  Synthetic Construct
ccccatggcgctcaggggtgcgtcggacgcc

SEQ ID NO: 17 - DNA, 27 bp, Artificial Sequence
  Synthetic Construct
acaggttggctgataagtccccggtct

SEQ ID NO: 18 - DNA, 33 bp, Artificial Sequence
  Synthetic Construct
ggatccagaccggggacttatcagccaacctgt

SEQ ID NO: 19 - DNA, 1047 bp, Chrysoperla carnea (Insect)
atggaaaaaaaggaatttcgtgttttgataaaatactgttttctgaagggaaaaaatacagtggaagcaaaaacttggcttgataatgagtttccggactctgccccagggaaatcaacaataattgattggtatgcaaaattcaagcgtggtgaaatgagcacggaggacggtgaacgcagtggacgcccgaaagaggtggttaccgacgaaaacatcaaaaaaatccacaaaatgattttgaatgaccgtaaaatgaagttgatcgagatagcagaggccttaaagatatcaaaggaacgtgttggtcatatcattcatcaatatttggatatgcggaagctctgtgcaaaatgggtgccgcgcgagctcacatttgaccaaaaacaacaacgtgttgatgattctgagcggtgtttgcagctgttaactcgtaatacacccgagtttttccgtcgatatgtgacaatggatgaaacatggctccatcactacactcctgagtccaatcgacagtcggctgagtggacagcgaccggtgaaccgtctccgaagcgtggaaagactcaaaagtccgctggcaaagtaatggcctctgtttttttcgatgcgcatggaataatttttatcgattatcttgagaagggaaaaaccatcaacagtgactattatatggcgttattggagcgtttgaaggtcgaaatcgcggcaaaacggccccatatgaagaagaaaaaagtgttgttccaccaagacaacgcaccgtgccacaagtcattgagaacgatggcaaaaattcatgaattgggcttcgaattgcttccccacccaccgtattctccagatctggcccccagcgactttttcttgttctcagacctcaaaaggatgctcgcagggaaaaaatttggctgcaatgaagaggtgatcgccgaaactgaggcctattttgaggcaaaaccgaaggagtactaccaaaatggtatcaaaaaattggaaggtcgttataatcgttgtatcgctcttgaagggaactatgttgaataa

SEQ ID NO: 20 - protein, 348 aa, Chrysoperla carnea (Insect)
MEKKENRVLIKYCNLKGKNTVEAKTWLDNENPDSAPGKSTIIDWYAKFKRGEMSTEDGERSGRPKEVVTDENIKKIHKMILNDRKMKLIEIAEALKISKERVGHIIHQYLDMRKLCAKWVPRELTNDQKQQRVDDSERCLQLLTRNTPENFRRYVTMDETWLHHYTPESNRQSAEWTATGEPSPKRGKTQKSAGKVMASVNFDAHGIINIDYLEKGKTINSDYYMALLERLKVEIAAKRPHMKKKKVLFHQDNAPCHKSLRTMAKIHELGFELLPHPPYSPDLAPSDNFLFSDLKRMLAGKKNGCNEEVIAETEAYNEAKPKEYYQNGIKKLEGRYNRCIALEGNYVE